(19) United States
(12) Patent Application Publication
(10) Pub. No.: US 2014/0350648 A1
(43) Pub. Date: Nov. 27, 2014

(54) BODY TEMPERATURE REDUCTION SYSTEMS AND ASSOCIATED METHODS

(71) Applicant: Dynasil Biomedical Corporation, Watertown, MA (US)

(72) Inventors: Daniel Grant Ericson, Rochester, MN (US); Kyle Robert Brandy, New Hope, MN (US); Paul Edward Glynn, Braintree, MA (US); Michael Edward O'Neill, Randolph, MA (US)

(73) Assignee: Dynasil Biomedical Corporation, Watertown, MA (US)

(21) Appl. No.: 14/365,390

(22) PCT Filed: Dec. 14, 2012
(86) PCT No.: PCT/US2012/069765

§ 371 Date: Jun. 13, 2014

Related U.S. Application Data
(60) Provisional application No. 61/576,645, filed on Dec. 16, 2011.

(51) Int. Cl. A61F 7/10

(52) U.S. Cl. CPC A61F 7/10; A61F 2007/0009
USPC 607/105

(57) ABSTRACT

Systems and methods for lowering the core body temperature of subject are generally described. In certain embodiments, the core body temperature of a subject can be lowered by using a heat exchanger configured to cool an intubation gas that is transported to the subject via an intubation tube. The intubation tube used to deliver cooled intubation gas to the subject can include one or more features facilitating cooling of the subject. For example, in certain embodiments, the intubation tube may include multiple lumens. In some embodiments, one of the lumens can be used to deliver the relatively cool intubation gas and a second lumen can be used to transport relatively warm gas away from the patient's lungs. In certain embodiments, the system can be configured such that water (e.g., in the form of ice particles and/or liquid mist) can be delivered to the subject via the intubation tube, which can provide an enhanced cooling effect.

### RELATED APPLICATIONS

This application claims priority under 35 U.S.C. §119(e) to U.S. Provisional Patent Application No. 61/576,645, filed Dec. 16, 2011, and entitled “Body Temperature Reduction Systems and Associated Methods,” which is incorporated herein by reference in its entirety for all purposes.

### TECHNICAL FIELD

Systems and methods for reducing body temperature or inducing hypothermia are generally described.

### BACKGROUND

Reducing the body's metabolism can decrease the amount of damage that metabolically active organs (e.g., the heart, the brain, etc.) sustain during ischemic and/or hypoxic events such as heart attacks and strokes. Accordingly, deliberate lowering of body temperature (i.e., inducing hypothermia) has been used in a variety of medical procedures including heart surgery, brain surgery, spinal surgery, organ transplantation procedures, and the like.

A variety of methods for lowering body temperature and inducing hypothermia are known in the art. Known methods include, for example, applying cold cloth or sponges to the body, applying ice packs to the body, submerging the body in cold fluid, and transporting a cooled gas mixture including helium to the lungs of the subject. Despite the benefits provided by the systems and methods known in the art, additional performance enhancements would be desirable.

### SUMMARY

Systems and methods for reducing body temperature, e.g. for inducing hypothermia, are described. The subject matter of the present invention involves, in some cases, interrelated products, alternative solutions to a particular problem, and/or a plurality of different uses of one or more systems and/or articles.

In one aspect, an intubation tube is provided. In certain embodiments, the intubation tube comprises a first lumen comprising an inlet end and a discharge end; a second lumen comprising an inlet end and a discharge end; and a valve associated with the first lumen configured to restrict the flow of fluid from outside the intubation tube into the discharge end of the first lumen and to allow fluid to flow from inside the first lumen out of the discharge end of the first lumen.

The intubation tube comprises, in some embodiments, a lumen comprising an inlet end and a discharge end; and a heat exchanger lumen associated with the first lumen, the heat exchanger lumen comprising a fluidic pathway configured to transfer heat from the first lumen out of the intubation tube.

In another aspect, a system for lowering the core body temperature of a subject is provided. In certain embodiments, the system comprises a heat exchanger comprising an intubation gas inlet fluidically connected to a source of intubation gas, and an intubation gas outlet, wherein the heat exchanger is configured to cool intubation gas passing through the heat exchanger. In some embodiments, the system comprises an intubation tube fluidically connected to the intubation gas outlet of the heat exchanger and fluidically connected to a source of a coolant having a boiling point of greater than about 37 degrees Celsius, the intubation tube comprising a discharge end configured to eject intubation gas into the airway of the subject.

In certain embodiments, a system for lowering the core body temperature of a subject comprises a heat exchanger comprising an intubation gas inlet fluidically connected to a source of intubation gas, and an intubation gas outlet, wherein the heat exchanger is configured to cool intubation gas passing through the heat exchanger. In some embodiments, the system comprises an intubation tube comprising a first lumen fluidically connected to the intubation gas outlet of the heat exchanger, the first lumen comprising a discharge end configured to eject intubation gas into the airway of the subject, and a valve positioned at or near the discharge end of the first lumen configured to restrict the flow of fluid from outside the intubation tube into the discharge end of the first lumen and to allow fluid to flow from inside the first lumen out of the discharge end of the first lumen.

In another aspect, a method of lowering the core body temperature of a subject is provided. The method comprises, in certain embodiments, transporting an intubation gas through a heat exchanger such that the intubation gas is cooled, and at least a portion of the cooled intubation gas is transported through an intubation tube to an airway of the subject; and transporting a coolant with a boiling point of greater than about 37 degrees Celsius through the intubation tube to the airway of the subject.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is an exemplary schematic illustration of a system for lowering the core body temperature of a subject, according to some embodiments;

FIGS. 2A-2E are, according to certain embodiments, exemplary schematic illustrations of intubation tubes used to deliver fluid to a subject;

FIGS. 3A-3D are exemplary schematic illustrations of a heat exchanger and an integrated system that can be used to cool an intubation gas for delivery to a subject, according to some embodiments;

FIGS. 4A-4E are exemplary schematic illustrations of intubation tubes, according to certain embodiments;

FIG. 5 is an exemplary schematic illustration of a system for lowering the core body temperature of a subject, according to one set of embodiments; and

FIG. 6 is an exemplary plot of temperature as a function of time, illustrating hypothermic cooling in a pig, according to one set of embodiments.

### DETAILED DESCRIPTION

Systems and methods for lowering the core body temperature of subject are generally described. While most of the discussion below focuses on the application of inducing hypothermia, it should be understood that certain embodiments of the invention may be used/practiced for reducing the core body temperature of a hyperthermic subject (e.g. one suffering from fever or heat stroke) as well. In certain embodiments, the core body temperature of a subject can be lowered by using a heat exchanger configured to cool an intubation gas that is transported to the subject via an intubation tube. The intubation tube used to deliver cooled intubation gas to the subject can include one or more features facilitating cooling of the subject. For example, in certain embodiments, the intubation tube may include multiple lumens. In some embodiments, one of the lumens can be used to deliver the relatively cool intubation gas and a second lumen can be used to transport relatively warm gas away from the patient's lungs. In certain embodiments, the system can be configured such that a fluid comprising water (e.g., in the form of ice particles and/or liquid mist) can be delivered to the subject via the intubation tube, which can provide an enhanced cooling effect.

The injection of cooled gas into a subject's lungs to decrease body temperature is known in the art. For example, U.S. Pat. No. 6,983,749 to Kumar et al. describes a method of lowering body temperature by transporting a cooled gas mixture including helium into the lungs of the subject. In previous cooling systems, however, the intubation gas is often not effective in achieving the desired level of cooling. For example, in certain previous systems, perfluorocarbons are transported to the subject's lung in order to effect cooling, which can provide effective cooling in part due to their low boiling point and associated latent heat of vaporization. However, perfluorocarbons have a number of shortcomings. For example, perfluorocarbons are generally very expensive (e.g., over $500 per liter). Gases such as helium can be used as a replacement, but helium may not in certain instances provide sufficient cooling. Described herein are inventive systems and methods that are, in certain embodiments, able to provide effective cooling using fluids with boiling points greater than about 37° C., such as water.

It has also been discovered, in the context of the present invention, that typical conventional intubation tubes are not ideally suited for subject cooling, and therefore, in certain embodiments of the invention, inventive intubation tubes are provided and used. In typical previous cooling systems, the intubation tube includes a single lumen that is used to transport the cooled gas into the subject and to transport gas warmed within the subject's lungs out of the subject's body. When a single lumen is used to transport gas into and out of the subject, a rewarming effect is generally observed, which can negate much if not all of the cooling effect. Specifically, it is believed that the cooled intubation gas entering the subject remixes with warm air being transported away from the airway of the subject, thereby re-heating the cooled intubation gas. In certain embodiments of the present invention, the intubation tube used to deliver cooled intubation gas to the subject can include multiple lumens. In some embodiments, a first lumen can be used to deliver the relatively cool intubation gas and a second lumen can be used to transport relatively warm gas away from the patient's lungs. By isolating the cooled gas from the relatively warm return gas, one can limit the extent to which the cooled fluid is reheated prior to reaching the lungs of the subject, thereby providing an enhanced cooling effect.

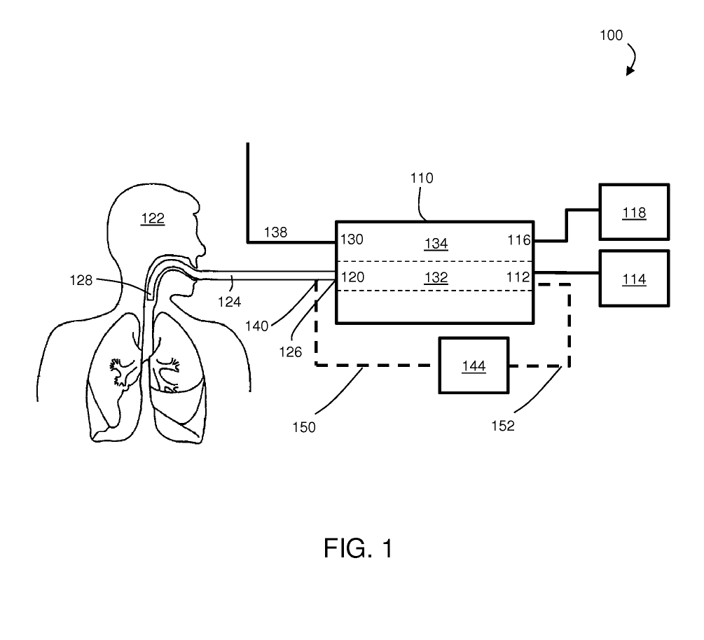

FIG. 1 is a schematic illustration of a system 100 for lowering the core body temperature of subject 122, according to some embodiments. System 100 includes heat exchanger 110 comprising an intubation gas inlet 112 fluidically connected to source 114 of intubation gas. Heat exchanger 110 can be configured to transfer heat from the intubation gas to another component of the heat exchanger, such as a heat exchanger coolant fluid, thereby cooling the intubation gas.

In some embodiments, the heat exchanger can be configured such that, once the intubation gas has been cooled, the intubation gas is delivered to the subject (e.g., via an intubation tube). In FIG. 1, heat exchanger 110 includes an intubation gas outlet 120 fluidically connected to inlet 126 of intubation tube 124. In some embodiments, the intubation tube comprises an endotracheal tube. In some such embodiments, the endotracheal tube can be configured to be inserted into the trachea of the subject, and the discharge end of the endotracheal tube can be configured to be positioned within the trachea of the subject during delivery of the intubation gas (and/or a supplemental coolant, e.g., having a boiling point of greater than about 37 degrees Celsius, as described in more detail below). For example, in FIG. 1, intubation tube 124 is configured such that discharge end 128 of intubation tube 124 ejects intubation gas into the airway (e.g., the trachea and eventually the lungs) of subject 122. In some such embodiments, intubation tube 124 can further comprise a balloon or other flexible material that can be inflated to seal one cavity or passageway within a subject (e.g., the lungs) from other cavities or passageways within the subject (e.g., the esophagus, the stomach, and the like).

The intubation gas can be cooled within the heat exchanger using a variety of suitable methods. In FIG. 1, for example, heat exchanger 110 includes a heat exchanger coolant fluid inlet 116 fluidically connected to a source 118 of heat exchanger coolant fluid. Heat exchanger 110 can be configured such that heat from the intubation gas is transported to the heat exchanger coolant fluid. In certain embodiments, the heat exchanger may be configured such that, once the intubation gas has been cooled, the heat exchanger coolant fluid is transported out of the heat exchanger. In alternative embodiments, the heat exchanger coolant fluid may be contained under essentially non-flow conditions, for example as in a cooled fluid bath. In other embodiments, the heat absorbing media may be in solid form, such as an ice block or cooled graphite block, metal block, solid component of a Peltier cooler, etc. As an example of a flow-through heat exchanger, in FIG. 1, heat exchanger 110 includes a heat exchanger coolant fluid outlet 130 from which the heat exchanger coolant fluid used to cool the intubation gas in the heat exchanger is expelled. Optionally, after the heat exchanger coolant fluid is transported out of the heat exchanger, it can be transported through conduit 138, purified and/or re-cooled, and transported back to source 118 for further use in system 100. In other embodiments, the heat exchanger coolant fluid can be directly vented after use in heat exchanger 110. One of ordinary skill in the art would be capable of identifying suitable heat exchanger coolant fluids, which can include liquids and/or gases. Examples include, but are not limited to, polyethylene glycol, methanol, glycerol, propylene glycol, ammonia, chlorofluorocarbons, hydrochlorofluorocarbons, hydrofluorocarbons, helium, oxygen, nitrogen, sulfur dioxide, a liquefied gas (e.g. liquefied nitrogen) and/or mixtures of these (e.g., air).

In some embodiments, in addition to the intubation gas, intubation tube 124 can be configured to deliver a supplemental coolant. For example, in some embodiments, intubation tube 124 can be configured to transport a supplemental coolant having a boiling point of greater than about 37 degrees Celsius. In certain embodiments, at least a portion of the supplemental coolant can undergo a phase change (e.g., melting, vaporization, etc.) within the subject to provide an additional cooling load.

The intubation gas and/or the supplemental coolant delivered to the subject can comprise a variety of components. In some embodiments, the intubation gas and/or the supplemental coolant comprises air or simulated air (i.e., a mixture of oxygen and nitrogen with an oxygen:nitrogen ratio of approximately a 20:80). In certain embodiments, the intubation gas and/or the supplemental coolant is substantially free of supplemental helium (e.g., the intubation gas and/or the supplemental coolant can contain helium in an amount of less than about 1%, less than about 0.1%, less than about 0.01% by volume, or can contain helium in an amount of 0% by volume). In some embodiments, the intubation gas and/or the supplemental coolant is substantially free of perfluorocarbons (e.g., the intubation gas and/or the supplemental coolant can contain perfluorocarbons in an amount of less than about 1%, less than about 0.1%, less than about 0.01% by volume, or can contain perfluorocarbons in an amount of 0% by volume). The ability to operate without the use of supplemental perflourocarbon(s) and/or supplemental helium can reduce system complexity and cost and allow one to avoid introducing compounds into the airway of the subject that are not naturally present within the subject. Of course, one of ordinary skill in the art would understand that the invention is not limited to such embodiments, and in other cases, one or more supplemental perfluorocarbons and/or supplemental helium could be employed.

As noted elsewhere, the supplemental coolant can contain a component having a boiling point of greater than 37° C. In one particularly advantageous set of embodiments, the supplemental coolant comprises H₂O. The H₂O can be in solid and/or liquid form. For example, in certain embodiments, the H₂O comprises ice, such as ice particles injected or otherwise transported into and/or within the intubation tube. In certain embodiments, the H₂O comprises liquid water. Liquid water can be transported into the intubation tube in the form of, for example, a mist of water droplets, a substantially continuous stream of water, or any other suitable form. In certain embodiments, the supplemental coolant can be added to the intubation tube and/or the heat exchanger in the liquid phase and can freeze within the intubation tube and/or the heat exchanger to form a solid phase (e.g., solid particles) prior to being delivered to the subject.

In certain embodiments, one or more salts or other additives can be included in the supplemental coolant (e.g., included in liquid water, solid ice, and/or any other suitable supplemental coolant), which can lower the freezing point of the supplemental coolant, thereby decreasing the temperature at which the desired phase change occurs and providing more effective cooling. Examples of suitable salts that can be included in the supplemental coolant include, for example, chloride salts (e.g., sodium chloride (NaCl), potassium chloride (KCl), calcium chloride (CaCl₂), magnesium chloride (MgCl₂)) and the like.

Supplemental coolant can be added to the intubation tube, to the heat exchanger (e.g., to the intubation gas inlet), or at any other suitable point in the system. For example, in the set of embodiments illustrated in FIG. 1, supplemental coolant source 144 is fluidically connected to intubation tube 124 via conduit 150. In certain embodiments, the source of coolant is configured to inject the coolant into the intubation tube at a location at or downstream of a location on the intubation tube that is fluidically connected to the intubation gas outlet of the heat exchanger. For example, in FIG. 1, coolant source 144 is configured to inject the supplemental coolant into the intubation tube at location 140, which is downstream of the location on intubation tube 124 that is fluidically connected to the intubation gas outlet 120 of heat exchanger 110. In some embodiments, supplemental coolant can be injected into intubation tube 124 at substantially the same location as the location on intubation tube 124 that is fluidically connected to the intubation gas outlet 120 of heat exchanger 110.

The supplemental coolant can also be delivered to the system at locations upstream of the location on the intubation tube that is fluidically connected to the intubation gas outlet of the heat exchanger, in addition to or in place of other delivery locations. For example, the supplemental coolant from source 144 can be transported to an inlet of heat exchanger 110 via conduit 152. In some such embodiments, the supplemental coolant can be transported through and cooled within the heat exchanger (e.g., heat exchanger 110 and/or another heat exchanger) prior to being transported to intubation tube 124. In some such embodiments in which heat exchanger 110 is used to pre-cool the supplemental coolant from source 144, heat exchanger 110 can comprise a separate coolant inlet and a separate coolant outlet for the supplemental coolant to be delivered to the subject. In some such embodiments, the supplemental coolant can be transported through heat exchanger 110 via a separate conduit which can, for example, be surrounded by second conduit 134 of heat exchanger 110.

In certain embodiments, supplemental coolant can be atomized prior to being transported to intubation tube 124. For example, in FIG. 1, an atomizer can be positioned at or near the discharge end (at or near location 140) of conduit 150 connecting source 144 to intubation tube 124 (or connecting source 144 to heat exchanger 110) and/or at the discharge end of conduit 152 connecting source 144 to heat exchanger 110. The atomizer can produce a mist of liquid and/or ice particles, prior to injecting the supplemental coolant and/or as the supplemental coolant is injected. In one particular set of embodiments, the atomizers comprise nozzles including 100 micrometer openings configured to produce liquid droplets (e.g., liquid water droplets) between 1 micrometer and 5 micrometers in diameter.

In one particular set of embodiments, a supplemental coolant comprising water can be used to generate ice particles for delivery to the subject via the intubation tube. For example, source 144 can comprise a container (e.g., a tank) in which water, saline, or other water-containing coolant is stored. The water-containing coolant can then be transported along conduit 152 for transport to an inlet of heat exchanger 110 (e.g., intubation gas inlet 112 or another heat exchanger inlet dedicated to receiving supplemental coolant). In certain embodiments, a programmable dispenser can be used, which can deliver a predetermined volume of water-containing coolant to the heat exchanger (e.g., for each cycle in a series of cycles). In certain embodiments, a mist of water-containing coolant can enter the heat exchanger in liquid form (e.g., at about 90° C.). In some embodiments, the water-containing mist within heat exchanger 110 can be cooled to below 0° C., thereby forming ice-containing particles. The ice-containing particles can subsequently be transported to an inlet of intubation tube 124 (e.g., the inlet through which intubation gas is transported into intubation tube 124 or another inlet (e.g., of a fluidically separated lumen) dedicated to receiving supplemental coolant). Of course, supplemental water-containing coolant can also be delivered directly to intubation tube 124 along conduit 150, in addition to or in place of the delivery along conduit 152. In some such embodiments, the water-containing coolant can be atomized at the discharge end of conduit 150 and, in some cases, form ice particles within intubation tube 124.

Supplemental coolant from source 144 can be transported through intubation tube 124 and delivered to the lungs via a lumen within intubation tube 124, as described in more detail below. In some embodiments, the system is configured to inject the intubation gas and the supplemental coolant (e.g., with a boiling point of greater than about 37 degrees Celsius) into a single lumen of intubation tube 124. In other embodiments, the system is configured to inject the intubation gas into one lumen of intubation tube 124 and to inject the supplemental coolant into a different lumen of intubation tube 124. For example, the lumen within intubation tube 124 that is used to deliver the supplemental coolant can be isolated from the lumen in intubation tube 124 used to deliver the intubation gas, in some cases, along substantially the entire length of the intubation tube.

In some embodiments in which the supplemental coolant is provided to the intubation tube in liquid form, at least a portion of the liquid supplemental coolant transported through the intubation tube may be atomized prior to and/or upon being delivered to the subject. For example, in some embodiments, one or more atomizers positioned at or near discharge end 128 of intubation tube 124 can be configured to atomize the supplemental coolant as it is ejected from the intubation tube. In one particular set of embodiments, the atomizers can comprise nozzles comprising 100 micrometer openings configured to produce liquid droplets (e.g., liquid water droplets) between 1 micrometer and 5 micrometers in diameter. By dispersing the liquid in small droplets prior to/upon delivering it to the subject, the speed at which the liquid is evaporated can be increased, which can lead to more rapid or effective cooling of the region of the subject to which the liquid is delivered.

In some embodiments, the inlet end of the intubation tube (e.g., the inlet end of a lumen of the intubation tube) can be positioned relatively close to the intubation gas outlet of the heat exchanger. For example, as illustrated in FIG. 1, the inlet end 126 of intubation tube 124 is in contact with intubation gas outlet 120 of heat exchanger 110, when the systems is configured for operation. Positioning the inlet end of the intubation tube relatively close to the intubation gas outlet of the heat exchanger can ensure that the intubation gas is not re-heated or only re-heated to a limited extent prior to being transported through the intubation tube. In some embodiments, the inlet end of at least one lumen of the intubation tube is positioned within about 10 centimeters, within about 5 centimeters, within about 1 centimeter, or within about 1 millimeter of the intubation gas outlet of the heat exchanger, when the systems is configured for operation. In one particular set of embodiments, the intubation tube is in direct connection to the intubation gas outlet of the heat exchanger when the system is configured for use by, for example, joining the intubation tube and the intubation gas outlet with a fitted connection (e.g., a threaded connection, a compression fit connection, or any other suitable connection).

In some embodiments, the intubation gas outlet of the heat exchanger can be positioned relatively close to the discharge end of the intubation tube. For example, in FIG. 1, intubation gas outlet 120 of heat exchanger 110 can be positioned relatively close to discharge end 128 of intubation tube 124. Positioning the intubation gas outlet of the heat exchanger relatively close to the discharge end of the intubation tube can advantageously ensure that the intubation gas is not excessively reheated prior to being administered to the subject. In some embodiments, the intubation gas outlet of the heat exchanger is positioned within 5 meters, within 1 meter, within 50 centimeters, or within 20 centimeters of the discharge end of the intubation tube, when the systems is configured for operation.

In certain embodiments, the heat exchanger used to cool the intubation gas can be positioned a short distance from the mouth of the subject. For example, in the set of embodiments illustrated in FIG. 1, heat exchanger 110 can be configured to be positioned a relatively short distance from the mouth of subject 122. Positioning the heat exchanger used to cool the intubation gas relatively closely to the mouth of the subject can advantageously ensure that the intubation gas is not excessively reheated prior to being delivered to the subject. In some embodiments, the heat exchanger can be positioned within 30 centimeters, within 20 centimeters, within 10 centimeters, or within 5 centimeters of the mouth of the subject, when the systems is configured for operation.

In some embodiments, at least one of the temperature and the pressure of a fluid (e.g., the intubation gas, a supplemental coolant, etc.) within the intubation tube can be measured, for example, prior to or as coolant is delivered to the subject. Measurement of a temperature or pressure can be achieved using, for example, one or more sensors integrated with the intubation tube, as described in more detail below. The ability to measure the temperature or pressure of a coolant being delivered to a subject can allow one to adjust upstream system parameters as necessary to provide an effective cooling load to the subject. In certain embodiments, both a temperature and pressure are able to be measured by the sensor(s).

In addition to inventive systems and methods for body temperature reduction, inventive intubation tubes are also described. In some embodiments, the intubation tube comprises a first lumen comprising an inlet end and a discharge end and a second lumen comprising an inlet end and a discharge end. In certain embodiments, the first lumen can be configured for transporting intubation gas from outside the subject to the airway of the subject, and the second lumen can be configured to transport fluid from the subject's airway to a location outside the subject. For example, in certain embodiments, the intubation tube can be configured such that fluid exiting the airway of the subject is restricted from being transported through the first lumen and thereby transported through the second lumen, while fluid (e.g., intubation gas) being transported to the airway of the subject is allowed to be transported through the first lumen.

Directionally selective transportation of fluids through the intubation tube can be achieved using a valve. In certain embodiments, the intubation tube comprises a valve associated with the first lumen. The valve can be configured to restrict the flow of fluid from outside the intubation tube (e.g., within the subject's airway) into the discharge end of the first lumen. In certain embodiments the valve can be configured to allow fluid to flow from inside the first lumen out of the discharge end of the first lumen (e.g., into the subject's airway). In this way, the valve can ensure that fluid is transported through the first lumen only in one direction (e.g., from outside the subject to the subject's airway).

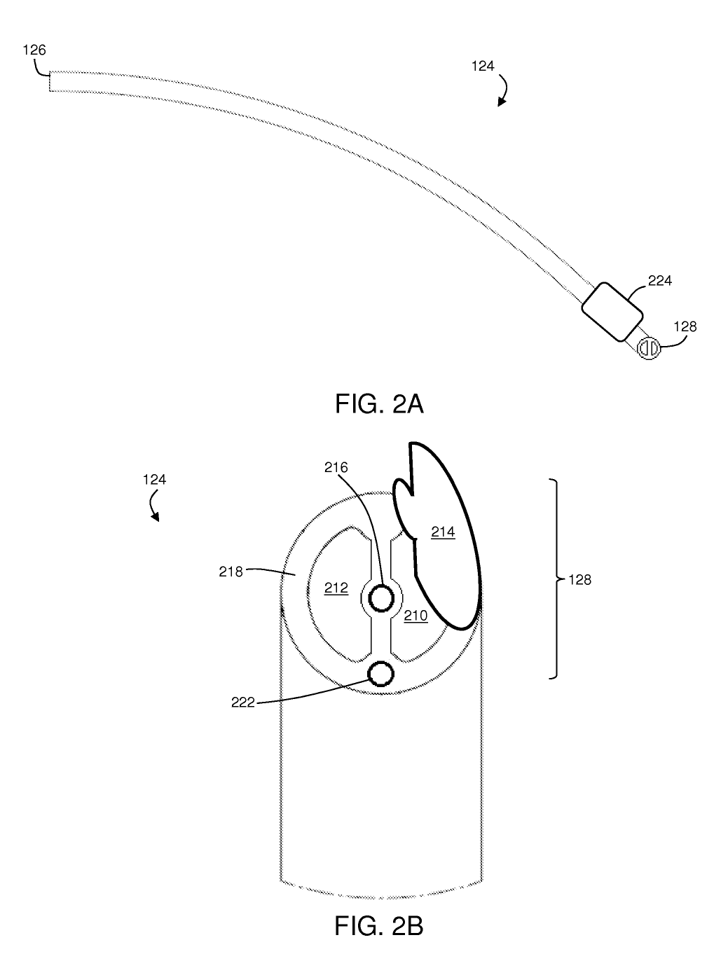

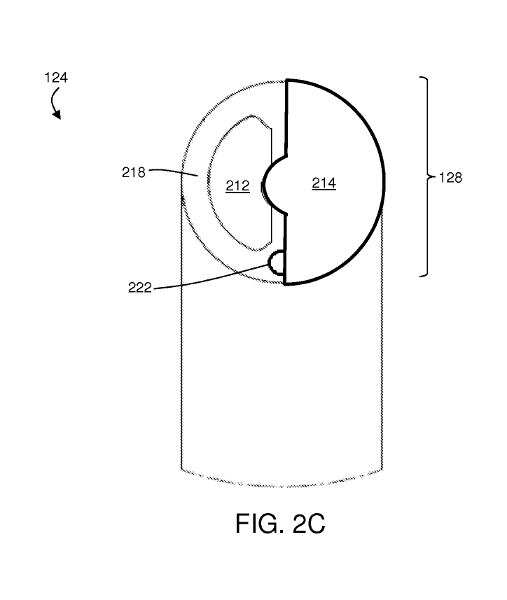

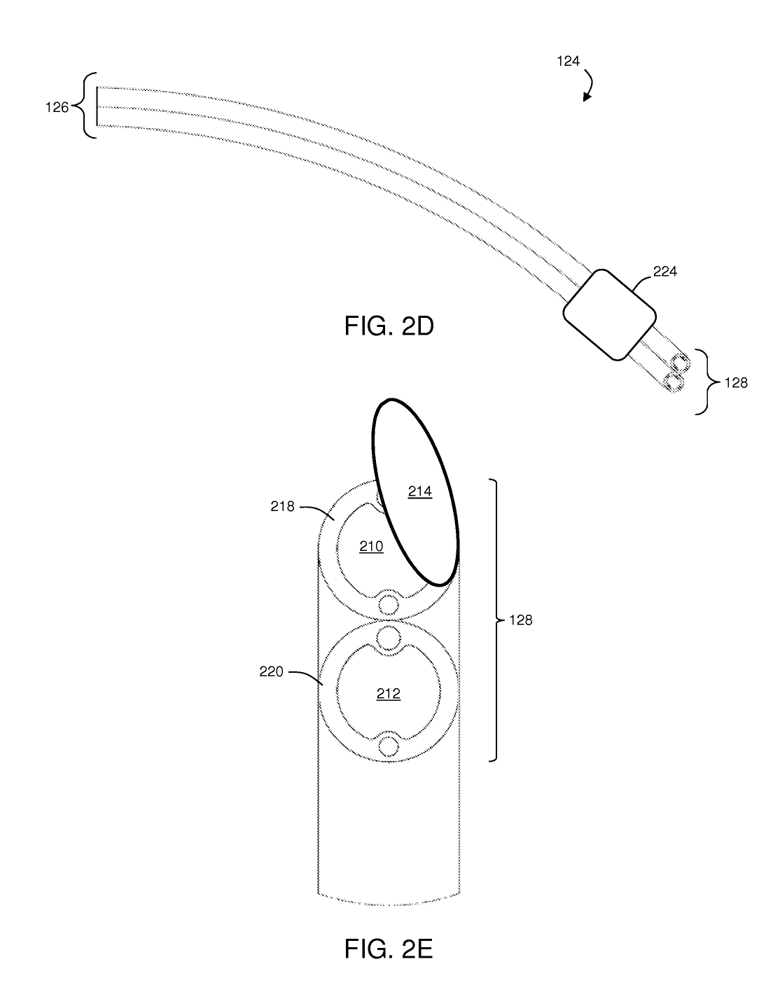

FIGS. 2A-2B are schematic illustrations of an exemplary intubation tube 124, which can be used in association with certain embodiments. FIG. 2A shows the entire length of intubation tube 124, while FIG. 2B is a close-up view of discharge end 128 of intubation tube 124. In FIGS. 2A-2B, intubation tube 124 includes first lumen 210 which can be configured to transport, for example, an intubation gas such as intubation gas from source 114 in FIG. 1. In addition, intubation tube 124 includes second lumen 212, which can be configured, for example, to transport a fluid from the airway of the subject (e.g., from lung(s) of the subject) out of the subject, for example, when the subject is exhaling. The discharge ends of the first lumen and the second lumen can be sized and configured to be inserted into an airway of a subject during use as illustrated, for example, in FIG. 1.

Intubation tube 124 can further comprise valve 214. Valve 214 can be associated with first lumen 210 and configured to restrict the flow of fluid from outside intubation tube 124 into the discharge end of first lumen 210. In addition, valve 214 can be configured to allow fluid to flow from inside first lumen 210 out of the discharge end of first lumen 210. Valve 214 can be positioned at any suitable point in or near intubation tube 124. In certain embodiments, valve 214 is positioned within first lumen 210. In some embodiments, valve 214 can be positioned at or near the discharge end 128 of intubation tube 124. For example, in the set of embodiments illustrated in FIG. 2B, valve 214 is positioned at the discharge end of lumen 210. In other embodiments, valve 214 can be positioned at or near the inlet end of intubation tube 124. Of course, valve 214 can be positioned at any location within lumen 210 to achieve the desired effect.

Valve 214 can be arranged in any suitable fashion. For example, in certain embodiments, valve 214 is configured to at least partially cover the discharge end of first lumen 210 to restrict the flow of fluid from outside intubation tube 124 into first lumen 210 and to at least partially uncover the discharge end of first lumen 210 to allow fluid to flow from inside first lumen 210 out of the discharge end of first lumen 210. One such valve is illustrated in FIG. 2B, which includes a flapper valve positioned at the discharge end of lumen 210. In FIG. 2B, valve 214 is illustrated in the open position, which can allow fluid to flow out of the discharge end of lumen 210 (for example, when intubation gas is being delivered to the airway of the subject). FIG. 2C illustrates valve 214 in the closed position. When arranged in this fashion, gas or other fluids can be restricted from entering lumen 210 (for example, when fluid is being transported from the airway of the subject out of the subject, such as when the subject is exhaling). Of course, any suitable valve can be used to control fluid flow into and/or out of lumen 210. For example, some embodiments, valve 214 comprises a flapper valve, a check valve, an electronic valve, and/or a ball valve.

Valve 214 can inhibit mixing of the cooled intubation gas (and/or supplemental coolant) with the re-warmed gas exhaled from the subject's airway. This can inhibit premature re-heating of the cooled fluid delivered to the subject's airway, enhancing the cooling effect.

In certain embodiments, mixing of the cooled intubation gas and the re-heated fluid exhaled from the airway can be further inhibited by isolating lumen 210 from lumen 212 along at least a portion of the length of intubation tube 124 such that the contents of the lumens do not mix or do so only to a limited extent. In FIG. 2B, first lumen 210 is fluidically isolated from second lumen 212 along substantially the entire length of intubation tube 124.

In certain embodiments, first lumen 210 can contain, flowing therethrough, a first fluid, and second lumen 212 can contain flowing therethrough a second fluid that is warmer than the first fluid. For example, in certain embodiments, lumen 210 can be configured to transport intubation gas, supplemental coolant (e.g., ice, liquid water, etc.), and/or another component used to lower the body temperature of the subject. In certain embodiments, lumen 212 can be configured to transport fluid that is being exhaled from the subject, which can be warmed within the airway of the subject during cooling of the subject.

The intubation gas and the supplemental coolant (e.g., including a water-containing liquid, ice-containing particles, etc.) can be transported to the subject within a single lumen, in certain embodiments. For example, in the set of embodiments illustrated in FIG. 2B, lumen 210 can be configured to transport both the intubation gas and the supplemental coolant. In other embodiments, separate lumens can be used to transport intubation gas and the supplemental coolant. For example, in FIG. 2B, intubation tube 124 can comprise third lumen 216. In certain embodiments, third lumen 216 can be configured to transport supplemental coolant while lumen 210 can be configured to transport intubation gas. Of course, in other embodiments, lumen 216 can be configured to transport intubation gas while lumen 210 is configured to transport supplemental coolant. In FIGS. 2B-2C, valve 214 is configured to control the flow into and out of both lumen 210 and lumen 216 (e.g., by covering and uncovering the lumen(s)). Of course, control of flow within lumen 216 is optional, and in other embodiments, valve 214 can be configured to control the flow into and out of lumen 210, but not into and out of lumen 216.

Lumen 216 can be used to transport a liquid such as liquid water and/or a solid such as ice particles (e.g., in combination with a carrying fluid). In certain embodiments, lumen 216 includes an atomizer at or near the discharge end of lumen 216, which can be used to atomize a liquid (e.g., liquid water, optionally including a freezing point depressant such as a salt or any other suitable liquid) that is transported out of lumen 216 prior to entry into the subjects airway. Optionally, intubation tube 124 can include one or more additional lumens for transporting additional coolants.

In FIGS. 2A-2C, first lumen 210 is configured as a first elongated orifice within tube body 218, and second lumen 212 is configured as a second elongated orifice within tube body 218. In other embodiments, other configurations are possible. For example, in FIGS. 2D-2E, first lumen 210 is configured as an elongated orifice within a first tube body 218, and second lumen 212 is configured as an elongated orifice within a second tube body 220 associated with the first tube body. In FIGS. 2D-2E, first tube body 218 is in contact with second tube body 220. First and second tube bodies 218 and 220 in FIGS. 2D-2E can be formed as separate tube bodies and subsequently joined, or they can be formed as a single unitary joined body.

Referring back to the set of embodiments illustrated in FIGS. 2A-2C, intubation tube 124 can optionally comprise an additional lumen 222. In FIGS. 2A-2C, lumen 222 is configured as an elongated orifice within tube body 218. Lumen 222 can be configured to house, for example, one or more sensors. The sensor(s) can be configured to measure at least one of a temperature and a pressure, for example, of a fluid within intubation tube 124. In some embodiments, the sensor(s) can be positioned within the intubation tube such that the sensor is within the subject during use of the intubation tube, which can allow, for example, one to measure a temperature and pressure of a fluid in the intubation tube during use. In certain embodiments, the sensor(s) can be configured to measure a pressure and/or temperature at or near the discharge end of the intubation tube (e.g., at or near the discharge end of any lumen within the intubation tube) during use. As one example, the sensor within lumen 222 can comprise a temperature sensor (e.g., a thermocouple) configured to measure a temperature of a fluid within intubation tube 124. In certain embodiments, the temperature sensor within lumen 210 and/or lumen 212.

The measurement made by the sensor within lumen 222 can be used to adjust a parameter within the system. For example, in certain embodiments, system 100 (in which intubation 124 can be used) is configured to adjust a flow rate and/or a temperature of the intubation gas and/or the supplemental coolant at the inlet end of intubation tube 124 based at least in part on the temperature and/or pressure determination made by the sensor(s) within lumen 222. As one particular example, a temperature sensor within lumen 222 can be used to measure the temperature of the coolant exiting the discharge end of lumen 210. If the gas exiting lumen 210 is too cold, system 100 can increase the temperature of the intubation gas and/or supplemental coolant and/or system 100 can lower the flow rate of the intubation gas and/or supplemental coolant transported through lumen 210. If the gas exiting lumen 210 is too warm, system 100 can reduce the temperature of the intubation gas and/or supplemental coolant and/or system 100 can increase the flow rate of the intubation gas and/or supplemental coolant transported through lumen 210.

Examples of temperature sensors that can be positioned within lumen 222 include, but are not limited to, thermocouples, resistive temperature sensors, infrared sensors, bimetallic devices, change of state sensors, and the like. Examples of pressure sensors that can be positioned within lumen 222 include, for example, piezoresistive strain gauges, capacity pressure sensors, electromagnetic pressure sensors, piezoelectric pressure sensors, optical pressure sensors, potentiometric pressure sensors, resonant pressure sensors, thermal pressure sensors, and the like. In some embodiments, electrochemical sensors (e.g., pH sensors), fiber optic sensors, and/or glucose sensors can be positioned within a lumen of the intubation tube. While a single lumen for housing a sensor is illustrated in FIGS. 2B-2C, in other embodiments, one or more additional lumens can be incorporated into the intubation tube, which can allow for the simultaneous placement of multiple sensors (e.g., multiple temperature sensors, multiple pressure, and or a combination of one or more temperature sensors and one or more pressure sensors).

In some embodiments, intubation tube 124 comprises an additional lumen (not illustrated in FIGS. 2B-2C), which can be configured to transport a gas for inflating a balloon or other flexible member. For example, in FIGS. 2A-2C, intubation tube 124 comprises a lumen fluidically connected to balloon 224 configured to transport an inflation fluid (e.g., a gas such as air) to balloon 224. The balloon or other flexible member can be configured to seal a first cavity or passageway in the subject (e.g., the lungs) from another cavity or passageway in the subject (e.g., the stomach, esophagus, etc.).

The intubation tubes described herein can be manufactured using a variety of methods. For example, in some embodiments, the intubation tube can be formed by extruding a material, such as a polymeric material, through a die to produce one or more tubes with multiple lumens. In some embodiments, multiple tubes can be attached (e.g., adhered or bonded). In some embodiments, first and second materials can be co-extruded such that the first material occupies the space defined by the material body and the second material occupies the space defined by the lumens. The second material can then be removed from the co-extruded body to form the final intubation tube structure. The intubation tubes described herein can be fabricated, in some embodiments, using hot melt tunneling, by forming a material (e.g., a melted polymer) over pre-positioned sensors or tubes, or any other methods known to those of ordinary skill in the art.

The material body of the intubation tube can be formed using a variety of materials. For example, in some embodiments, the material body of the intubation tube comprises one or more polymers (e.g., polyurethane, silicone, poly(vinyl chloride), polypropylene, polyethylene, polyesters, and/or polyamides), metals (e.g., copper, aluminum, and the like), or combinations of two or more of these materials.

Referring back to FIG. 1, heat exchanger 110 can assume a variety of configurations. In some embodiments, heat exchanger 110 comprises a first conduit 132 and a second conduit 134. In some embodiments, the first conduit 132 of heat exchanger 110 is disposed within the second conduit 134 of heat exchanger 110, for example in a shell and tube arrangement. In some embodiments, multiple conduits are disposed within second conduit 134 in a shell and tube arrangement. In some embodiments, second conduit 134 is configured such that it the longitudinal axis of second conduit 134 is substantially parallel to the longitudinal axes of the conduits contained within it (e.g., first conduit 132).

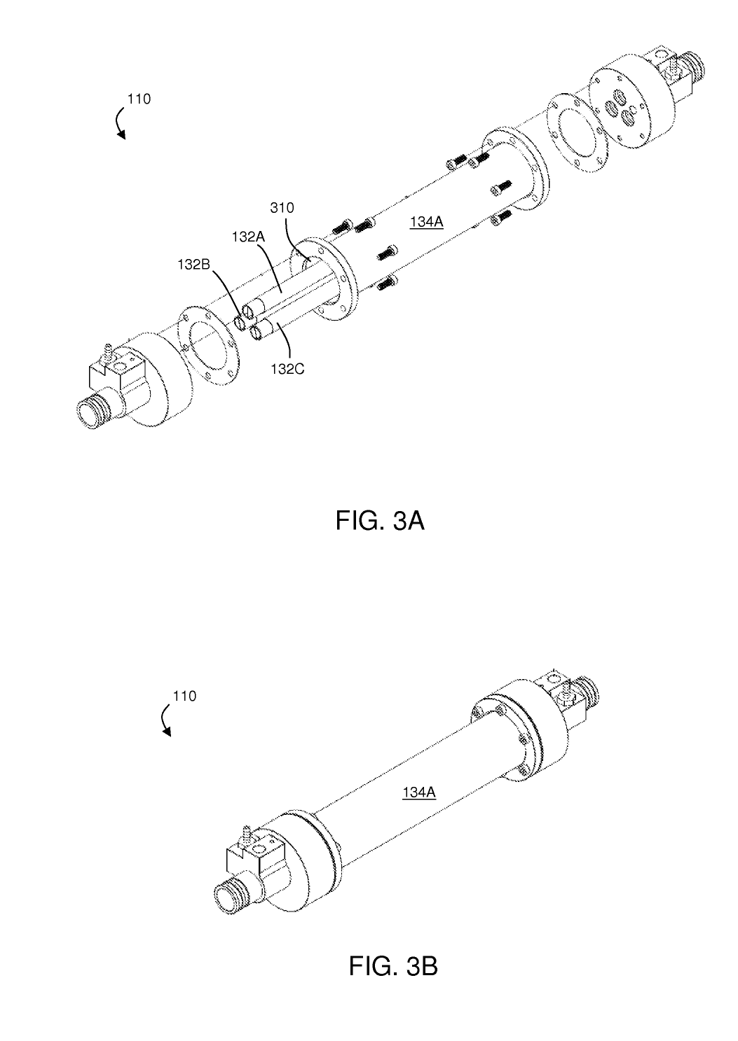

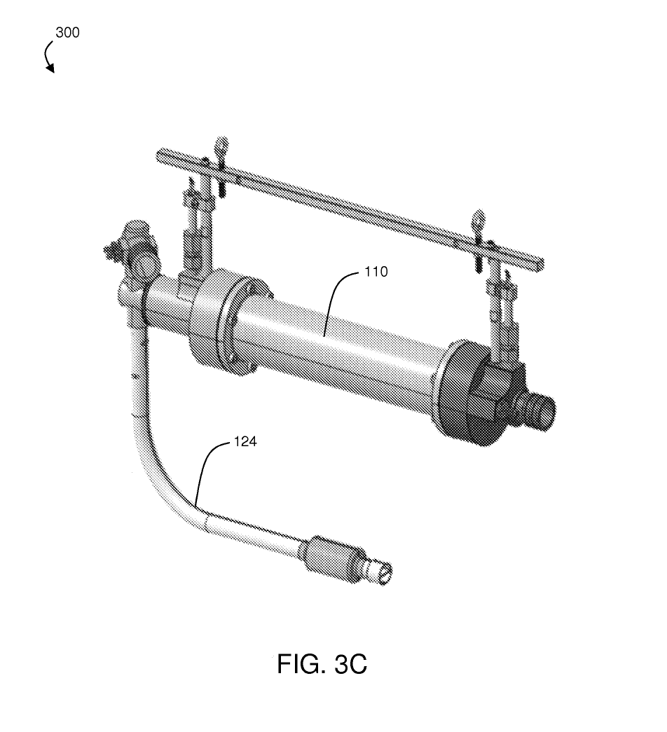

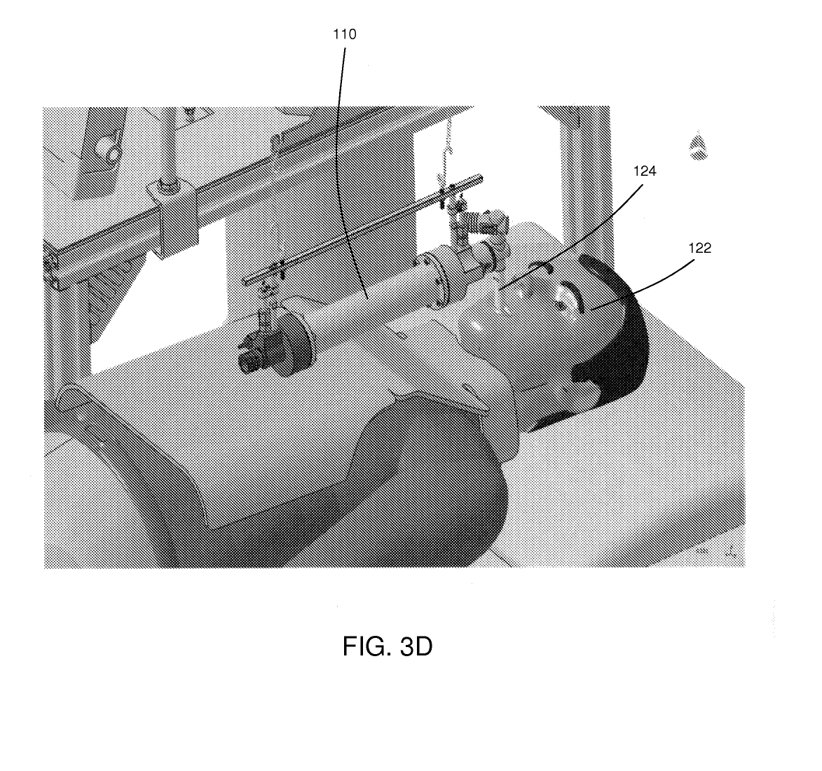

FIGS. 3A-3B are schematic illustrations of an exemplary heat exchanger 110, which can be used to transfer heat from an intubation gas (and, in some cases, a supplemental coolant) to a coolant fluid or other heat sink. FIG. 3A is a schematic of a disassembled heat exchanger, while FIG. 3B is a schematic illustration of the assembled heat exchanger. As illustrated in FIG. 3A, heat exchanger 110 includes a plurality of inner conduits 132A, 132B, and 132C, each of which is disposed within outer conduit 134A to form a shell and tube heat exchanger. In FIG. 3A, the longitudinal axes of each of inner conduits 132A, 132B, and 132C are substantially parallel to the longitudinal axis of outer conduit 134A. The heat exchanger can be configured, in some cases, such that intubation gas is transported through conduits 132A, 132B, and 132C while cooling fluid is transported through outer conduit 134A (e.g., via inlet 310). In other cases, the heat exchanger can be configured such that cooling fluid is transported through conduits 132A, 132B, and 132C while intubation gas is transported through outer conduit 134A. In addition, in some embodiments, the heat exchanger can be configured such that a liquid (e.g., a refrigerant) is transported through at least one of conduits 132A, 132B, 132C, and/or 134A.

FIG. 3C is a schematic illustration of a system 300 in which heat exchanger 110 is integrated with intubation tube 124. As illustrated in FIG. 3C, heat exchanger 110 and intubation tube 124 are directly connected using a threaded fitting. FIG. 3D is a schematic illustration of system 300 in which the discharge end intubation tube 124 has been positioned within the airway of subject 122. As illustrated in FIG. 3D, heat exchanger 110 is positioned a relatively short distance (e.g., less than 10 centimeters) from the mouth of subject 122. In other embodiments, the heat exchanger can be positioned even closer to the mouth of subject 122 during use (and in some cases, can be in contact with the subject during use).

The conduits of the heat exchanger can be formed from a variety of materials. In some embodiments, the inner conduits include materials with relatively high thermal conductivities to enhance the rate at which heat is transferred between the coolant fluid and the intubation gas. For example, all or part of the inner conduits can be formed of a metal or metals such as aluminum, copper, steel (e.g. stainless steel), titanium, alloys of these or other metals, and the like.

While three inner conduits are illustrated in FIG. 3A, it should be understood that, in other embodiments, more or fewer inner conduits may be present. For example, in some embodiments, the heat exchanger may comprise a single inner conduit or two inner conduits housed within a single outer conduit. In some embodiments, the heat exchanger may comprise at least 4, at least 5, at least 10, or more inner conduits housed within an outer conduit.

Fluid may be transported through heat exchanger 110 according to a variety of configurations. In some embodiments, the intubation gas and the coolant fluid can be flowed through heat exchanger 110 in a co-current flow configuration. In other embodiments, the coolant fluid and the intubation gas can be transported through the heat exchanger 110 in a counter-current configuration. In addition, one or more baffles, fins, or other fluid-directing components may be integrated into one or more conduits within heat exchanger 110 to direct the flow of fluid.

It should be understood that a standalone heat exchanger (e.g., heat exchanger 110 in FIGS. 1 and 3A-3D) is not required for operation of the system. In certain embodiments, for example, intubation tube 124 comprises a heat exchanger. The heat exchanger can be integrated with intubation tube 124 such that, during use, at least a portion of the heat exchanger is positioned within the airway of the subject. The integrated heat exchanger of intubation tube 124 can be used in place of or in addition to stand alone heat exchanger 110.

In some embodiments, the heat exchanger of the intubation tube comprises a fluidic pathway configured to transfer heat from a lumen of the intubation tube (e.g., from an intubation gas and/or a supplemental coolant within a lumen) out of the intubation tube. By arranging the heat exchanger in this manner, the intubation gas and/or supplemental coolant can be cooled as it is transported through the intubation tube.

The heat exchanger of the intubation tube can be arranged in a variety of configurations. In some embodiments, the heat exchanger can comprise one or more lumens through which a heat exchanger coolant fluid can be transported. In certain embodiments, the intubation tube can be configured to include at least one lumen that transports heat exchanger coolant fluid. In certain arrangements, the intubation tube can be configured to transport heat exchanger coolant fluid from an inlet end of the intubation tube to a location at or near the discharge end of the intubation tube. In some embodiments, the intubation tube can be configured such that when the heat exchanger coolant fluid reaches a location at or near the discharge end of the intubation tube the direction of flow of the heat exchanger coolant fluid is altered such that the heat exchanger coolant fluid is returned towards or to the inlet and of the intubation tube.

For example, in certain embodiments, the fluidic pathway of the heat exchanger comprises a jacket surrounding at least a portion of the lumen of the intubation tube. In some embodiments, the intubation tube heat exchanger can be in the form of a separate lumen associated with the intubation tube.

Heat exchange can be achieved, for example, by transporting a heat exchanger coolant into and out of the fluidic pathway of the heat exchanger. Any suitable heat exchanger fluid could be used within the integrated heat exchanger, including, for example, polyethylene glycol, methanol, glycerol, propylene glycol, ammonia, chlorofluorocarbons, hydrochlorofluorocarbons, hydrofluorocarbons, helium, oxygen, nitrogen, sulfur dioxide, a liquefied gas (e.g. liquefied nitrogen) and/or mixtures of these (e.g., air). In certain embodiments, the heat exchanger fluid within intubation tube 124 has a freezing point lower than about 0° C. or lower than about −15° C. (e.g., about −20° C.).

In some embodiments, the heat exchanger can be an integrated part of the intubation tube, such that the heat exchanger and the intubation tube form a monolithic structure. In other embodiments, the heat exchanger can be formed separately from the intubation tube and subsequently associated with the intubation tube (e.g., via an adhesive, mechanical fasteners, or other suitable attachment or by coiling tubing for carrying the heat exchanger coolant around the intubation tube, etc.).

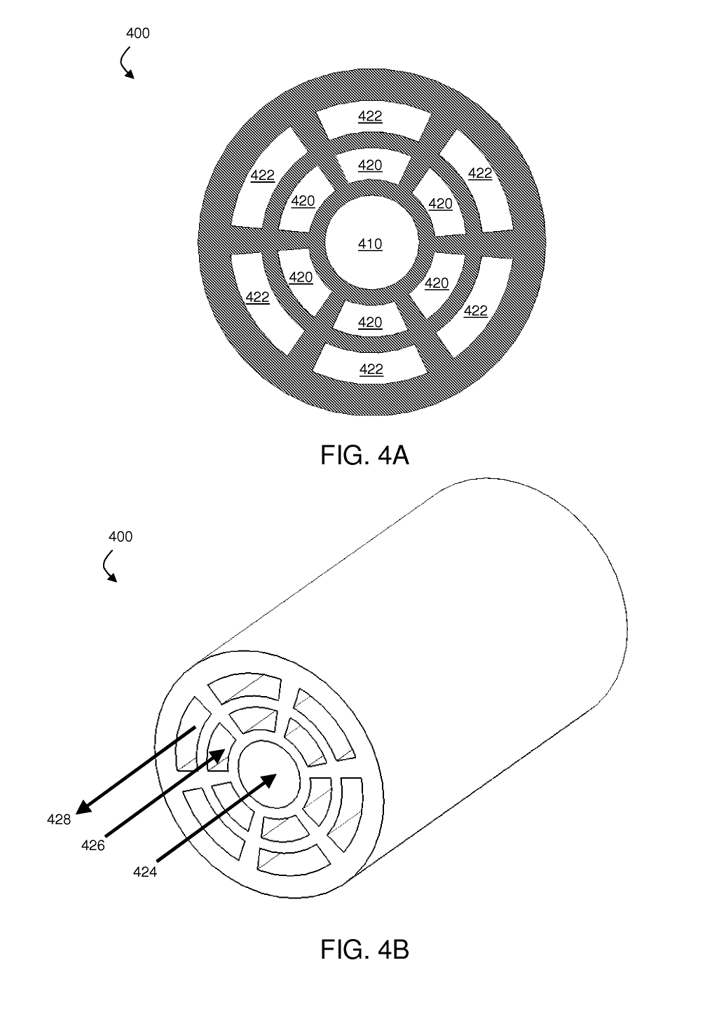

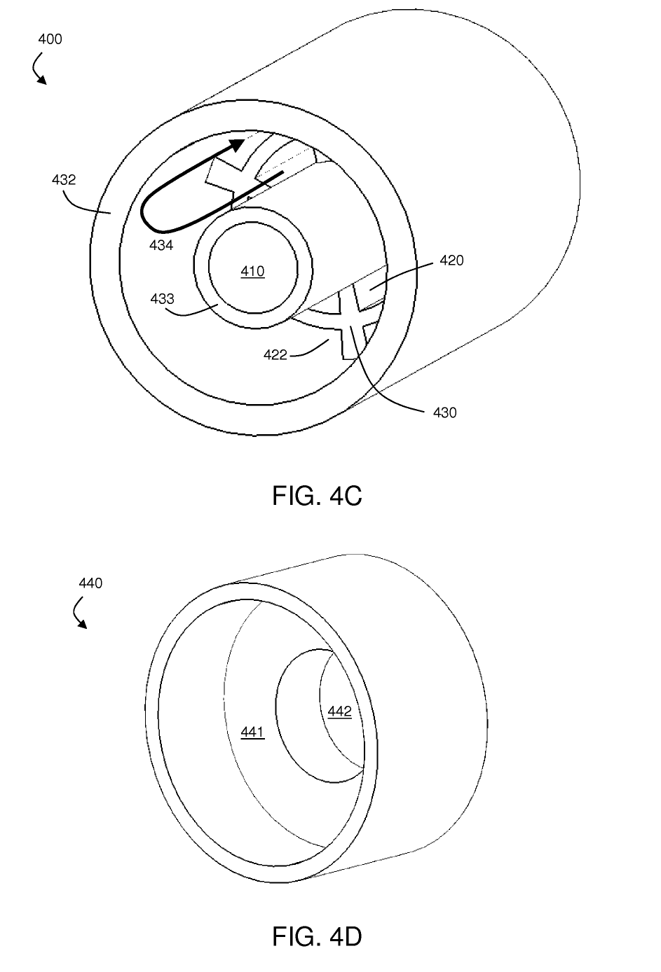

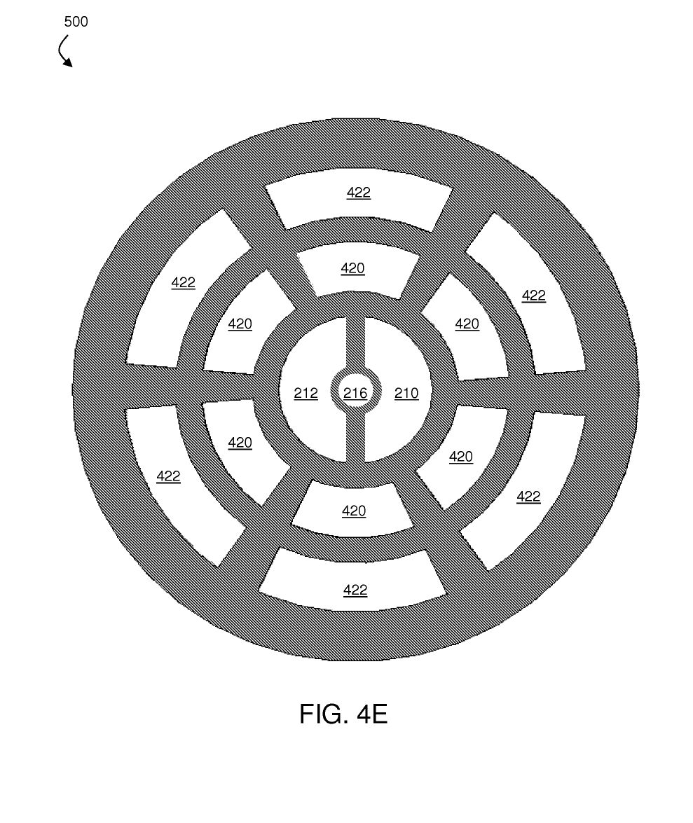

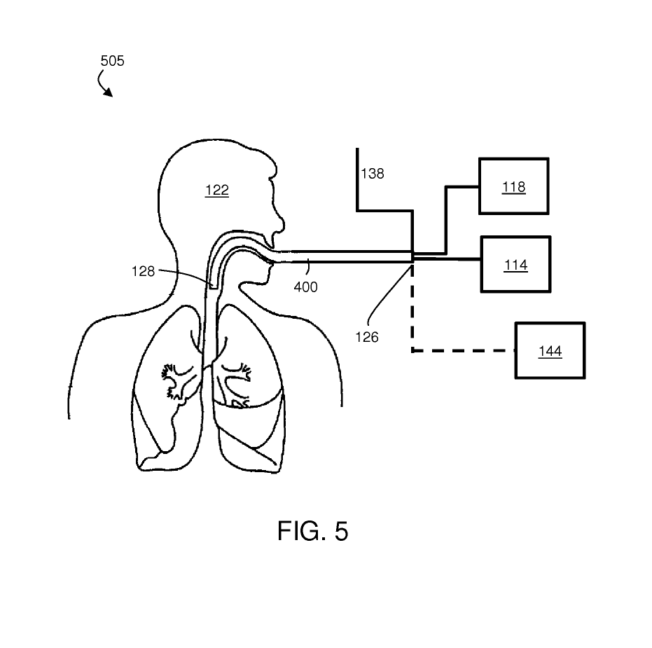

FIGS. 4A-4D are schematic illustrations of an intubation tube comprising an integrated heat exchanger, according to one set of embodiments. Intubation tube 400 includes lumen 410, which can be used to transport an intubation gas or other intubation fluid to a subject. Tube 400 also includes a plurality of lumens 420. Lumens 420 can be configured to transport a heat exchanger coolant fluid from the inlet end of the intubation tube to a location at or near the discharge end of the intubation tube. In addition, intubation tube 400 can include a plurality of lumens 422. Lumens 422 can be configured to transport the heat exchanger coolant fluid (e.g., the fluid transported into tube 400 via lumens 420) from a location at or near the discharge end of the intubation tube toward the inlet end and out of the intubation tube.

FIG. 4B is a perspective schematic illustration of the inlet end of tube 400, illustrating the flow pathway for the intubation gas and a heat exchanger coolant fluid. In FIG. 4B, intubation gas can be transported into lumen 410 along pathway 424. In addition, heat exchanger coolant fluid can be transported into lumen 420 via pathway 426. The heat exchanger coolant fluid can be used to cool fluid within lumen 410. For example, a relatively cold fluid can be transported into the intubation tube via lumens 420 and remove heat from the intubation fluid within lumen 410 prior to the intubation fluid being delivered to the airway of the subject. After the heat exchanger coolant fluid has cooled the intubation fluid within lumen 410, the heat exchanger coolant fluid can double back and be transported out of intubation tube via lumens 422, for example, via pathway 428 in FIG. 4B.

FIG. 4C is a perspective schematic illustration of the discharge end of intubation tube 400. As illustrated in FIG. 4C, walls 430 defining lumens 420 and 422 terminate prior to reaching end 432 of intubation tube 400. Accordingly, in FIG. 4C, there is a region between the area in which walls 430 terminate and tube end 432 in which the direction of flow of the heat exchanger coolant fluid can be reversed (e.g. via use of a structure such as the cap described below) so that it is transported back toward the inlet and an intubation tube 400. The reversal of flow of heat exchanger coolant fluid is illustrated as flow pathway 434 in FIG. 4C.

To ensure that the heat exchanger coolant fluid is not delivered to the airway of patient, the fluidic pathway of the heat exchanger coolant fluid may be sealed at the discharge end of tube 400. The discharge end of the heat exchanger coolant fluid pathway can be sealed by integrally forming (e.g. via injection molding) a wall (not shown) at the discharge end of the intubation tube that prevents liquid discharged from lumen 420 and/or 422 from being discharged from the discharge end of the intubation tube, while not preventing discharge from lumen 410, in some embodiments. In certain embodiments, the discharge end of the heat exchanger coolant fluid pathway can be sealed by positioning a cap at the discharge end of the intubation tube. An exemplary cap 440 is illustrated in FIG. 4D. The cap in FIG. 4D includes surface 441, which can be configured to produce a seal with walls 432 and 433 in FIG. 4C, thereby preventing heat exchanger coolant fluid from entering the airway of the subject. In addition, cap 440 can include passageway 442, which can be aligned with lumen 410 to allow intubation gas or other intubation fluids to be transported out of the discharge end of intubation tube 400 and into the airway of the subject.

While intubation tube 400 is illustrated as including a single lumen for the delivery of intubation gas or other intubation fluids, in other embodiments, the intubation tube comprising a heat exchanger can include multiple lumens for the delivery of intubation gas or other intubation fluids. For example, FIG. 4E is a schematic cross-sectional illustration of an intubation tube 500 including a first lumen 210 (e.g., for the delivery of intubation fluid from outside the tube to the airway of the subject) and a second lumen 212 (e.g., for the transport of intubation fluid and/or other fluid expired from the subject from the airway of the subject out of the subject). Intubation tube 500 can, in some embodiments, also include a lumen 216, which can be configured to transport supplemental coolant for delivery to the subject. Although not illustrated in FIG. 4E, intubation tube 500 can also include a valve (e.g., a valve similar to valve 214 in FIG. 2B) to provide control of the direction of fluid flow within lumens 210 and 212.

FIG. 5 is a schematic illustration of a system 505, illustrating the use of intubation tube 400, according to one set of embodiments. In contrast to system 100 in FIG. 1, system 505 does not require a standalone heat exchanger 110 (although in certain embodiments one could be used for supplemental cooling, if desired). In system 505 in the configuration as illustrated, intubation tube 400 includes an integrated heat exchanger. In FIG. 5, source 118 of heat exchanger coolant fluid and intubation gas source 114 are connected to inlet 126 of intubation tube 400. Optionally, a supplemental coolant can be transported to inlet 126 of intubation tube 400 from supplemental coolant source 144. In the set of embodiments illustrated in FIG. 5, cooling of the intubation gas can be achieved along the length of intubation tube 400, eliminating the need for a standalone heat exchanger.

Of course, in other embodiments, a standalone heat exchanger (e.g., heat exchanger 110 in FIG. 1) can be used in association with an intubation tube comprising a heat exchanger. In some such embodiments, heat exchanger coolant fluid outlet 130 of heat exchanger 110 can be fluidically connected to inlet end 126 of the intubation tube. In other such embodiments, a separate heat exchanger coolant fluid can be transported through the heat exchanger in the intubation tube.

While the systems herein have been described primarily for use with a human subject, it should be understood that in other embodiments non-human subjects can be used. For example, systems such as those described and outlined in FIGS. 1, 3C-3D, and 4B can be used on animals such as dogs, cats, horses, cows, pigs, and the like.

While intubation tubes have been described primarily for use in association with the systems and methods for lowering the core body temperature of a subject, as described elsewhere herein, use of the intubation tubes described herein is not so limited, and one of ordinary skill in the art would recognize that the intubation tubes described herein can be used in a variety of other systems and for a variety of other purposes, particularly where it is advantageous to deliver both a gas and a supplemental coolant (e.g., a coolant having a boiling point of greater than about 37 degrees Celsius) to the airway of a subject and in situations where pressure or temperature monitoring of a fluid delivered to the subject is desired.

The articles, systems, and methods described herein can be used in association with a variety of procedures in which it is useful to lower the body temperature of a subject. For example, the articles, systems, and methods described herein can be used to reduce the adverse impacts of reduced oxygen availability during a variety of ischemic events including, but not limited to, cardiac arrest, stroke, traumatic brain or spinal cord injury, neurogenic fever, and neonatal encephalopathy. The articles, systems, and methods described herein can also be used to treat, for example, heat stroke.

U.S. Provisional Patent Application No. 61/576,645, filed Dec. 16, 2011, and entitled “Body Temperature Reduction Systems and Associated Methods” is incorporated herein by reference in its entirety for all purposes.

The following example is intended to illustrate certain embodiments of the present invention, but does not exemplify the full scope of the invention.

### Example

This example describes transpulmonary evaporative cooling in swine using a mircoparticle cold air/ice mist to effectively induce therapeutic hypothermia. Thermoelectric induced cold mist was shown to promote swift heat extraction from blood circulating through the lungs. The result was the rapid lowering of the core temperature, using the lungs as heat exchange organs.

One female pig (Chester White, Swine), weighing 91 kg was used in this set of experiments. The temperature of the environment in which the experiments were performed was set to 70-72° F. Anesthesia was induced using intramuscular injections of ketamine (10 mg/kg) and xylazine (1 mg/kg). Following induction of anesthesia, peripheral intravenous catheters were inserted and lactated Ringers solution was infused at 125 ml/hour. The general anesthesia was deepened with thiopental (3 mg/kg) and pancuronium (0.1 mg/kg).

After the onset of neuromuscular paralysis, a specially designed intubation tube with an inside wall cooling track (similar to the intubation tube illustrated in FIGS. 4A-4B) was inserted into the trachea. Cooling was achieved by positioning the discharge end of the intubation tube within the lungs of the pig, and transporting cooled gas through the intubation tube to the lungs of the pig. In addition, ice mist was transported through a central lumen of the intubation tube, into the lungs of the pig. The ice mist was created by positioning the output end of a nebulizer at the input end of the heat exchanger; the water mist from the nebulizer was frozen to form ice mist upon entering the input of the heat exchanger, which was maintained at −25° C. The discharge end of the intubation tube was positioned at the carina between the left and right lung so as to allow proximity of the cooled fluid to the lungs.

The fluid within the intubation tube was cooled using a heat exchanger similar in configuration to the heat exchanger illustrated in FIGS. 1 and 3A-3D. The heat exchanger system could be both volume controlled and pressure controlled, with varying rates of free gas flow. The inspiratory limb of the breathing circuit was connected in series to the heat exchanger, which was capable of cooling the inspiratory gases to −25° C.

The pig was ventilated with oxygen-enriched room air (FiO₂ 0.21-0.4) so as to maintain arterial oxygen saturation above 90%, a tidal volume of 6-10 ml/kg, and a respiratory rate of 12-16 breaths per minute to maintain a PaCO₂ of 35-40 torr. Total intravenous anesthesia was maintained with propofol (1 mg/kg bolus, followed by an infusion of 75 μg/kg/minute), fentanyl (25 mg/kg bolus, followed by an infusion of 0.1-0.3 μg/kg/minute), and pancuronium (0.1 mg/kg/hour). Radiant heating lamps and warming blankets were used to maintain normothermia until the induction of hypothermia.

The pig was monitored using: EKG, pulse oximeter, blood pressure cuff, arterial line, pulmonary artery catheter and Foley catheter. Temperature probes were inserted into the rectum, ear, nasopharynx and esophagus in addition to thermistors already incorporated in the pulmonary artery catheter and Foley catheter. A fiberoptic transducer tipped pressure/temperature catheter was introduced into the brain parenchyma through a small right parietal bore hole for intracranial pressure and temperature measurements. The concentration of oxygen in the inspired gas mixture was adjusted as needed to avoid hypoxemia (SaO₂<90%).

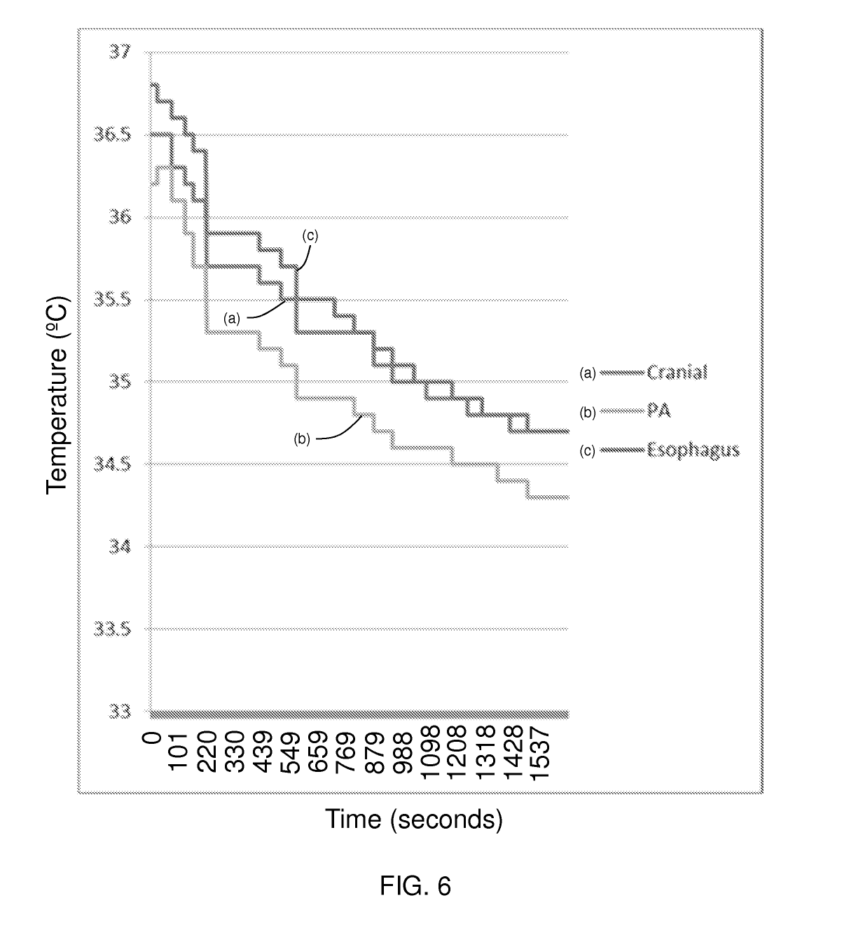

FIG. 6 is a plot of temperature as a function of time in the esophagus, cranium, and pulmonary artery. As seen in FIG. 6, hypothermic cooling was found to drop the cranial, pulmonary artery and esophageal temperatures to less than 35° C. within 30 minutes of initiation of the heat exchanger that was attached to the customized endotracheal tube. The coolant temperature at the end of the endotracheal tube was −25° C.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An intubation tube, comprising:
a first lumen comprising an inlet end and a discharge end;
a second lumen comprising an inlet end and a discharge end; and
a valve associated with the first lumen configured to restrict the flow of fluid from outside the intubation tube into the discharge end of the first lumen and to allow fluid to flow from inside the first lumen out of the discharge end of the first lumen.

2. The intubation tube of claim 1, wherein the valve is positioned within the first lumen.

3. The intubation tube of claim 1, wherein the valve is positioned at or near the discharge end of the intubation tube.

4. The intubation tube of claim 1, wherein the valve is positioned at or near the inlet end of the intubation tube.

5. The intubation tube of claim 1, wherein the valve comprises a check valve.

6. The intubation tube of claim 1, wherein the valve is configured to at least partially cover the discharge end of the first lumen to restrict the flow of fluid from outside the intubation tube into the first lumen and to at least partially uncover the discharge end of the first lumen to allow fluid to flow from inside the first lumen out of the discharge end of the first lumen.

7. The intubation tube of claim 6, wherein the valve comprises a flapper valve.

8. The intubation tube of claim 1, wherein the valve comprises an electronic valve.

9. The intubation tube of claim 1, wherein the valve comprises a ball valve.

10. The intubation tube of any one of claims 1-9, wherein the first lumen is fluidically isolated from the second lumen along substantially the entire length of the intubation tube.

11. The intubation tube of any one of claims 1-10, wherein the first lumen is contains flowing therethrough a first fluid, and the second contains flowing therethrough a second fluid that is warmer than the first fluid.

12. The intubation tube of any one of claims 1-11, wherein the first lumen is contains flowing therethrough a fluid comprising ice.

13. The intubation tube of any one of claims 1-12, wherein the discharge ends of the first lumen and the second lumen are sized and configured to be inserted into an airway of a subject during use.

14. The intubation tube of any one of claims 1-13, wherein the first lumen comprises a first elongated orifice within a tube body and the second lumen comprises a second elongated orifice within the tube body.

15. The intubation tube of any one of claims 1-14, wherein the first lumen comprises an elongated orifice within a first tube body and the second lumen comprises an elongated orifice within a second tube body associated with the first tube body.

16. The intubation tube of claim 15, wherein the first tube body is in contact with the second tube body.

17. The intubation tube of any one of claims 1-16, comprising a third lumen comprising an inlet end and a discharge end.

18. The intubation tube of claim 17, comprising an atomizer located at or near the discharge end of the third lumen.

19. The intubation tube of any one of claims 1-18, comprising a sensor integrated with the intubation tube and constructed and arranged to measure at least one of a temperature and a pressure at at least one location along the length of the intubation tube.

20. The intubation tube of claim 19, wherein the sensor is configured to measure a temperature at or near the discharge end of the first and/or second lumen.

21. A system for lowering the core body temperature of a subject, comprising:
a heat exchanger comprising:
an intubation gas inlet fluidically connected to a source of intubation gas, and
an intubation gas outlet,
wherein the heat exchanger is configured to cool intubation gas passing through the heat exchanger; and
an intubation tube fluidically connected to the intubation gas outlet of the heat exchanger and fluidically connected to a source of a coolant having a boiling point of greater than about 37 degrees Celsius, the intubation tube comprising a discharge end configured to eject intubation gas into the airway of the subject.

22. The system of claim 21, wherein the coolant having a boiling point of greater than about 37 degrees Celsius comprises H₂O.

23. The system of claim 22, wherein the source of coolant is configured to inject ice particles into the intubation tube.

24. The system of claim 22, wherein the source of coolant is configured to inject liquid water into the intubation tube.

25. The system of any one of claims 21-24, wherein the source of coolant is configured to inject the coolant into the intubation tube at a location at or downstream of a location on the intubation tube that is fluidically connected to the intubation gas outlet of the heat exchanger.

26. The system of any one of claims 21-25, wherein the system is configured to inject the intubation gas and the coolant with a boiling point of greater than about 37 degrees Celsius into a single lumen of the intubation tube.

27. The system of any one of claims 21-25, wherein the system is configured to inject the intubation gas into one lumen of the intubation tube and to inject the coolant with a boiling point of greater than about 37 degrees Celsius into a different lumen of the intubation tube.

28. The system of any one of claims 21-27, wherein an inlet end of a first lumen of the intubation tube is positioned within about 10 centimeters of the intubation gas outlet of the heat exchanger, when the system is configured for operation.

29. The system of any one of claims 21-28, wherein an inlet end of a first lumen of the intubation tube is positioned within about 5 centimeters of the intubation gas outlet of the heat exchanger, when the system is configured for operation.

30. The system of any one of claims 21-28, wherein an inlet end of a first lumen of the intubation tube is positioned within about 1 centimeter of the intubation gas outlet of the heat exchanger, when the system is configured for operation.

31. The system of any one of claims 21-28, wherein an inlet end of a first lumen of the intubation tube is positioned within about 1 millimeter of the intubation gas outlet of the heat exchanger, when the system is configured for operation.

32. The system of any one of claims 21-31, wherein the intubation tube comprises a first lumen having an inlet end and a discharge end and a second lumen having an inlet end and a discharge end.

33. The system of claim 32, wherein the first lumen is fluidically isolated from the second lumen along substantially the entire length of the intubation tube.

34. The system of any one of claims 32-33, wherein the first lumen is configured to transport the intubation gas, and the second lumen is configured to transport fluid from an airway of the subject.

35. The system of claim 34, wherein the second lumen is configured to transport fluid from a lung of the subject.

36. The system of any one of claims 21-35, wherein the intubation tube comprises a sensor integrated with the intubation tube and constructed and arranged to determine at least one of a temperature and a pressure at at least one location along the length of the intubation tube.

37. The system of claim 36, wherein the sensor is configured to determine a temperature at or near the discharge end of the intubation tube.

38. The system of claim 37, wherein the system is configured to adjust a flow rate and/or a temperature of the intubation gas and/or the coolant at the inlet end of the intubation tube based at least in part on the temperature determination.

39. A system for lowering the core body temperature of a subject, comprising:
a heat exchanger comprising:
an intubation gas inlet fluidically connected to a source of intubation gas, and
an intubation gas outlet,
wherein the heat exchanger is configured to cool intubation gas passing through the heat exchanger; and
an intubation tube comprising a first lumen fluidically connected to the intubation gas outlet of the heat exchanger, the first lumen comprising:
a discharge end configured to eject intubation gas into the airway of the subject, and
a valve positioned at or near the discharge end of the first lumen configured to restrict the flow of fluid from outside the intubation tube into the discharge end of the first lumen and to allow fluid to flow from inside the first lumen out of the discharge end of the first lumen.

40. The system of claim 39, wherein the valve comprises a check valve.

41. The system of claim 39, wherein the valve is configured to at least partially cover the discharge end of the first lumen to restrict the flow of fluid from outside the intubation tube into the first lumen and to at least partially uncover the discharge end of the first lumen to allow fluid to flow from inside the first lumen out of the discharge end of the first lumen.

42. The system of claim 41, wherein the valve comprises a flapper valve.

43. The system of any one of claims 39-42, wherein an inlet end of the first lumen of the intubation tube is positioned within about 10 centimeters of the intubation gas outlet of the heat exchanger, when the system is configured for operation.

44. The system of any one of claims 39-42, wherein an inlet end of the first lumen of the intubation tube is positioned within about 5 centimeters of the intubation gas outlet of the heat exchanger, when the system is configured for operation.

45. The system of any one of claims 39-42, wherein an inlet end of the first lumen of the intubation tube is positioned within about 1 centimeter of the intubation gas outlet of the heat exchanger, when the system is configured for operation.

46. The system of any one of claims 39-42, wherein an inlet end of the first lumen of the intubation tube is positioned within about 1 millimeter of the intubation gas outlet of the heat exchanger, when the system is configured for operation.

47. The system of any one of claims 39-46, wherein the intubation tube comprises a second lumen having an inlet end and a discharge end.

48. The system of claim 47, wherein the first lumen is fluidically isolated from the second lumen along substantially the entire length of the intubation tube.

49. The system of any one of claims 47-48, wherein the first lumen is configured to transport the intubation gas, and the second lumen is configured to transport fluid from an airway of the subject.

50. The system of claim 49, wherein the second lumen is configured to transport fluid from a lung of the subject.

51. The system of any one of claims 39-50, wherein the intubation tube comprises a sensor integrated with the intubation tube and constructed and arranged to measure at least one of a temperature and a pressure at at least one location along the length of the intubation tube.

52. The system of claim 51, wherein the sensor is configured to measure a temperature at or near the discharge end of the intubation tube.

53. The system of claim 52, wherein the system is configured to adjust a flow rate and/or a temperature of the intubation gas at the inlet end of the intubation tube based at least in part on the temperature determination.

54. A method of lowering the core body temperature of a subject, comprising:
transporting an intubation gas through a heat exchanger such that the intubation gas is cooled, and at least a portion of the cooled intubation gas is transported through an intubation tube to an airway of the subject; and
transporting a coolant with a boiling point of greater than about 37 degrees Celsius through the intubation tube to the airway of the subject.

55. The method of claim 54, wherein the coolant comprises H₂O.

56. The method of claim 55, wherein the coolant comprises ice particles.

57. The method of claim 55, wherein the coolant comprises liquid water.

58. The method of any one of claims 54-57, wherein the intubation gas is substantially free of supplemental helium.

59. The method of any one of claims 54-58, wherein the intubation gas is substantially free of perfluorocarbons.

60. The method of any one of claims 54-59, comprising transporting the intubation gas and the coolant to the airway of the subject via a first lumen.

61. The method of claim 60, comprising transporting a fluid from the airway of the subject out of the subject via a second lumen.

62. The method of any one of claims 54-59, comprising transporting the intubation gas to the airway of the subject via a first lumen and transporting the coolant to the airway of the subject via a second lumen.

63. The method of claim 62, comprising transporting a fluid from the airway of the subject out of the subject via a third lumen.

64. The method of claim 54, wherein the subject is a human subject.

65. The method of claim 54, comprising determining a temperature of the intubation gas and/or the coolant at or near a discharge end of the intubation tube, and adjusting a flow rate and/or a temperature of the intubation gas and/or the coolant at or near an inlet end of the intubation tube based at least in part upon the temperature determination.

66. An intubation tube, comprising:
a lumen comprising an inlet end and a discharge end; and
a heat exchanger lumen associated with the first lumen, the heat exchanger lumen comprising a fluidic pathway configured to transfer heat from the first lumen out of the intubation tube.

67. The intubation tube of claim 66, wherein the fluidic pathway of the heat exchanger comprises a jacket surrounding at least a portion of the lumen of the intubation tube.

68. The intubation tube of claim 66, wherein the fluidic pathway of the heat exchanger comprises a second lumen that extends along at least a portion of the length of the intubation tube.
